(19) United States
(12) Patent Application Publication
(10) Pub. No.: US 2003/0105171 A1
(43) Pub. Date: Jun. 5, 2003

(54) Modified zirconia support for catalyst for Fischer-Tropsch process

(75) Inventors: Munirpallam A. Subramanian, Kennett Square, PA (US); Norman Herron, Newark, DE (US); Stephan Schwarz, Wilmington, DE (US); Sergej A. Maslov, Ponca City, OK (US); Nithya Srinivasan, Ponca City, OK (US)

Correspondence Address: DAVID W. WESTPHAL, CONOCOPHILLIPS COMPNAY, P.O. BOX 1267, PONCA CITY, OK, 74602-1267, US

(73) Assignee: Conoco Inc., Houston, TX

(21) Appl. No.: 10/291,271

(22) Filed: Nov. 8, 2002

Related U.S. Application Data
(60) Provisional application No. 60/336,941, filed on Nov. 8, 2001.

(51) Int. Cl. C07C 27/06

(52) U.S. Cl. 518/715

(57) ABSTRACT

A process for producing hydrocarbons by the Fischer-Tropsch process is provided in which a cobalt-containing catalyst is supported on a modified zirconia support selected from among silica-zirconia, tungstated zirconia, and sulfated zirconia. The catalyst shows improved performance of up to 70% in the Fischer-Tropsch reaction as compared to a corresponding catalyst supported on unmodified zirconia.

### CROSS-REFERENCE TO RELATED APPLICATIONS

Not Applicable.

### STATEMENT REGARDING FEDERALLY SPONSORED RESEARCH OR DEVELOPMENT

Not Applicable.

### TECHNICAL FIELD OF THE INVENTION

The present invention relates generally to the field of Fischer-Tropsch reactions for the catalytic production of hydrocarbons from synthesis gas, a mixture of carbon monoxide and hydrogen. More particularly, the present invention relates to the use of a catalyst supported on a modified zirconia support, preferably selected from among silica-zirconia, sulfated zirconia, and tungstated zirconia.

### BACKGROUND OF THE INVENTION

Natural gas, found in deposits in the earth, is an abundant energy resource. For example, natural gas commonly serves as a fuel for heating, cooking, and power generation, among other things. The process of obtaining natural gas from an earth formation typically includes drilling a well into the formation. Wells that provide natural gas are often remote from locations with a demand for the consumption of the natural gas.

Thus, natural gas is conventionally transported large distances from the wellhead to commercial destinations in pipelines. This transportation presents technological challenges due in part to the large volume occupied by a gas. Because the volume of a gas is so much greater than the volume of a liquid containing the same number of gas molecules, the process of transporting natural gas typically includes chilling and/or pressurizing the natural gas in order to liquefy it. However, this contributes to the final cost of the natural gas.

Further, naturally occurring sources of crude oil used for liquid fuels such as gasoline and middle distillates have been decreasing and supplies are not expected to meet demand in the coming years. Middle distillates typically include heating oil, jet fuel, diesel fuel, and kerosene. Fuels that are liquid under standard atmospheric conditions have the advantage that in addition to their value, they can be transported more easily in a pipeline than natural gas, since they do not require the energy, equipment, and expense required for liquefaction.

Thus, for all of the above-described reasons, there has been interest in developing technologies for converting natural gas to more readily transportable liquid fuels, i.e. to fuels that are liquid at standard temperatures and pressures. One method for converting natural gas to liquid fuels involves two sequential chemical transformations. In the first transformation, natural gas or methane, the major chemical component of natural gas, is reacted with oxygen to form syngas, which is a combination of carbon monoxide gas and hydrogen gas. In the second transformation, known as the Fischer-Tropsch process, carbon monoxide is reacted with hydrogen to form organic molecules containing carbon and hydrogen. Those organic molecules containing only carbon and hydrogen are known as hydrocarbons. In addition, other organic molecules containing oxygen in addition to carbon and hydrogen are known as oxygenates and may be formed during the Fischer-Tropsch process. Hydrocarbons having carbons linked in a straight chain known as aliphatic hydrocarbons, which may include paraffins and/or olefins. Paraffins are particularly desirable as the basis of synthetic diesel fuel.

Typically the Fischer-Tropsch product stream contains hydrocarbons having a range of numbers of carbon atoms, and thus having a range of molecular weights. Thus, the Fischer-Tropsch products produced by conversion of natural gas commonly contain a range of hydrocarbons including gases, liquids and waxes. Depending on the molecular weight product distribution, different Fischer-Tropsch product mixtures are ideally suited to different uses. For example, Fischer-Tropsch product mixtures containing liquids may be processed to yield gasoline, as well as heavier middle distillates. Hydrocarbon waxes may be subjected to an additional processing step for conversion to liquid and/or gaseous hydrocarbons. Thus, in the production of a Fischer-Tropsch product stream for processing to a fuel it is desirable to maximize the production of high value liquid and/or wax hydrocarbons, such as hydrocarbons with at least 5 carbon atoms per hydrocarbon molecule (C₅₊ hydrocarbons).

Typically, in the Fischer-Tropsch synthesis, the distribution of weights that is observed such as for C₅₊ hydrocarbons, can be described by likening the Fischer-Tropsch reaction to a polymerization reaction with an Anderson-Shultz-Flory chain growth probability (α) that is independent of the number of carbon atoms in the lengthening molecule. α is typically interpreted as the ratio of the mole fraction of Cₙ₊₁ product to the mole fraction of Cₙ product. A value of α of at least 0.72 is preferred for producing high value liquid and/or wax hydrocarbons, such as C₅₊ hydrocarbons.

The Fischer-Tropsch process is commonly facilitated by a catalyst. Catalysts desirably have the function of increasing the rate of a reaction without being consumed by the reaction. A feed containing carbon monoxide and hydrogen is typically contacted with a catalyst in a reaction zone that may include one or more reactors.

The composition of a catalyst influences the relative amounts of hydrocarbons obtained from a Fischer-Tropsch catalytic process. Common catalysts for use in the Fischer-Tropsch process contain at least one metal from Group VIII of the Periodic Table (in the old IUPAC notation, which is used throughout the present specification).

Fischer-Tropsch catalysts have typically been prepared by depositing the active metal and any promoters on a support. The support is typically a porous material that provides mechanical strength. The support further provides a high surface area per amount of catalytic metal. Catalyst supports for catalysts used in Fischer-Tropsch synthesis of hydrocarbons have typically been refractory oxides (e.g., silica, alumina, titania, zirconia or mixtures thereof).

It has been thought that the support typically does not effect the performance of a catalyst. However, supports that can improve the performance of a catalyst are desirable, because such supports would lessen or eliminate the need for costly precious metal promoters. Thus, there remains a need for alternative Fischer-Tropsch supported catalysts.

### SUMMARY OF THE INVENTION

The present invention features converting a feed stream comprising carbon monoxide and hydrogen to a product stream comprising hydrocarbons in the presence of a catalyst that includes (a) a catalytically active metal, preferably cobalt, (b) an optional promoter (c) a modified zirconia support, preferably selected from among silica-zirconia, sulfated zirconia, and tungstated zirconia. The hydrocarbons produced preferably include hydrocarbons in the diesel weight range. For example, the hydrocarbons produced may include C₁₁⁺ hydrocarbons. According to a preferred embodiment, in a process for producing hydrocarbons, the catalyst has an improved performance, such as productivity and paraffin/olefin ratio, and the like, with respect to a corresponding catalyst that includes an unmodified zirconia support. Likewise, according to a preferred embodiment, the catalyst has an improved property, such as reducibility, dispersion, acidity, and the like, with respect to a corresponding catalyst that includes an unmodified zirconia support.

According to any one of the above-described embodiments, an improvement in performance is preferably at least 5%, more preferably at least 10%, still more preferably at least 30%, yet more preferably at least 70%. Likewise, an improvement in a property is preferably at least 5%, more preferably at least 10%, still more preferably at least 30%, yet more preferably at least 70%

An improvement in performance is computed as follows. The performance of a corresponding catalyst is subtracted from the performance of a catalyst according to any one of the herein-described embodiments of the present invention to obtain a difference. The difference is divided by the performance of the corresponding catalyst to obtain a ratio. This ratio is the improvement in performance, which may be specified as a fraction, or, as above, as a percent.

Likewise, an improvement in a property is computed as follows. The property of a corresponding catalyst is subtracted from the property of a catalyst according to any one of the herein-described embodiments of the present invention to obtain a difference. The difference is divided by the property of the corresponding catalyst to obtain a ratio. This ratio is the improvement in property, which may be specified as a fraction, or, as above, as a percent.

A corresponding catalyst is herein defined as a catalyst with a nominal composition substantially the same to within 10% of all species apart from the support, including catalytically active metal and any promoter or promoters.

Thus, the present invention comprises a combination of features and advantages which enable it to overcome various problems of prior processes. The various characteristics described above, as well as other features, will be readily apparent to those skilled in the art upon reading the following detailed description of the preferred embodiments of the invention.

### DETAILED DESCRIPTION OF THE PREFERRED EMBODIMENT

It has been discovered that the modification of a zirconia support for a cobalt-containing Fischer-Tropsch catalyst significantly improves the performance of the catalyst, as compared to the same catalyst using an unmodified zirconia support. Modifications of zirconia that may be used include doping with SiO₂, doping with WO₃, and doping with SO₄. Performance may be, for example, either or both of C₁₁₊ productivity and an increase in the paraffin/olefin ratio of the produced hydrocarbons. Likewise, it is believed that the modification of a zirconia support for a cobalt-containing Fischer-Tropsch catalyst significantly improves the properties of the catalyst, as compared to the same catalyst using a zirconia support. Properties may be any one or combination of example, reducibility, dispersion, acidity.. In particular, it has been found that, for some catalysts, improvement in performance and/or a property of at least 5%, more preferably at least 10%, still more preferably at least 30%, yet more preferably at least 70% is achieved.

Catalyst

The present catalyst preferably includes a catalytic metal. The catalytic metal is preferably a Fischer-Tropsch catalytic metal. In particular, the catalytic metal is preferably selected from among the Group 8 metals, such as iron (Fe), ruthenium (Ru), and osmium (Os), Group 9 metals, such as cobalt (Co), rhodium (Rh), and iridium (Ir), Group 10 elements, such as nickel (Ni), palladium (Pd), and platinum (Pt), and the metals molybdenum (Mo), rhenium (Re), and tungsten (W). The catalytic metal is more preferably selected from the iron-group metals (i.e. cobalt, iron, and nickel), and combinations thereof. The catalytic metal still more preferably is selected from among cobalt and iron. The catalyst preferably contains a catalytically effective amount of the catalytic metal. The amount of catalytic metal present in the catalyst may vary widely.

When the catalytic metal is cobalt, the catalyst preferably has a nominal composition that includes cobalt in an amount totaling from about 1% to 50% by weight (as the metal) of total catalyst composition (catalytic metal, support, and any optional promoters), more preferably from about 5% to 40% by weight, still more preferably from about 10 to about 37 wt. % cobalt, sill yet more preferably from about 15 to about 35 wt. % cobalt. It will be understood that % indicates percent throughout the present specification.

It will be understood that, when the catalyst includes more than one supported metal, the catalytic metal, as termed herein, is the primary supported metal present in the catalyst. The primary supported metal is preferably determined by weight, that is the primary supported metal is preferably present in the greatest % by weight.

The catalytic metal contained by a catalyst according to a preferred embodiment of the present invention is preferably in a reduced, metallic state before use of the catalyst in the Fischer-Tropsch synthesis. However, it will be understood that the catalytic metal may be present in the form of a metal compound, such as a metal oxide, a metal hydroxide, and the like. The catalytic metal is preferably uniformly dispersed throughout the support. It is also understood that the catalytic metal can be also present at the surface of the support, in particular on the surface or within a surface region of the support, or that the catalytic metal can be non-homogeneously dispersed onto the support.

Optionally, the present catalyst may also include at least one promoter known to those skilled in the art. The promoter may vary according to the catalytic metal. A promoter may be an element that also, in an active form, has catalytic activity, in the absence of the catalytic metal. Such an element will be termed herein a promoter when it is present in the catalyst in a lesser wt. % than the catalytic metal.

A promoter preferably enhances the performance of the catalyst. Suitable measures of the performance that may be enhanced include selectivity, activity, stability, lifetime, reducibility and resistance to potential poisoning by impurities such as sulfur, nitrogen, and oxygen. A promoter is preferably a Fischer-Tropsch promoter, that is an element or compound that enhances the performance of a Fischer-Tropsch catalyst in a Fischer-Tropsch process.

It will be understood that as contemplated herein, an enhanced performance of a catalyst may be calculated according to any suitable method known to one of ordinary skill in the art. In particular, an enhanced performance may be given as a percent and computed as the ratio of the performance difference to the performance of a reference catalyst. The performance difference is between the performance of the improved catalyst and the reference catalyst, where the reference catalyst is a similar corresponding catalyst having the nominally same amounts, e.g. by weight percent, of all components except the promoter. It will further be understood that as contemplated herein, a performance may be measured in any suitable units. For example, when the performance is the productivity, the productivity may be measured in grams product per hour per liter reactor volume, grams product per hour per kilogram catalyst, and the like.

Suitable promoters vary with the catalytic metal and may be selected from Groups 1-15 of the Periodic Table of the Elements. A promoter may be in elemental form. Alternatively, a promoter may be present in an oxide compound. Further, a promoter may be present in an alloy containing the catalytic metal. Except as otherwise specified herein, a promoter is preferably present in an amount to provide a weight ratio of elemental promoter: elemental catalytic of from about 0.00005:1 to about 0.5:1, preferably, from about 0.0005:1 to about 0.01:1 (dry basis).

Further, when the catalytic metal is cobalt, suitable promoters include Group 1 elements such as potassium (K), lithium (Li), sodium (Na), and cesium (Cs), Group 2 elements such as calcium (Ca), magnesium (Mg), strontium (Sr), and barium (Ba), Group 3 elements such as scandium (Sc), yttrium (Y), and lanthanum (La), Group 4 elements such as (titanium) (Ti), zirconium (Zr), and hafnium (Hf), Group 5 elements such as vanadium (V), niobium (Nb), and tantalum (Ta), Group 6 elements such as molybdenum (Mo) and tungsten (W), Group 7 elements such as rhenium (Re) and manganese (Mn), Group 8 elements such as ruthenium (Ru) and osmium (Os), Group 9 elements such as rhodium (Rd) and iridium (Ir), Group 10 elements such as platinum (Pt) and palladium (Pd), Group 11 elements such as silver (Ag) and copper (Cu), Group 12 elements, such as zinc (Zn), cadmium (Cd), and mercury (Hg), Group 13 elements, such as gallium (Ga), indium (In), thallium (Tl), and boron (B), Group 14 elements such as tin (Sn) and lead (Pb), and Group 15 elements such as phosphorus (P), bismuth (Bi), and antimony (Sb). When the catalytic metal is cobalt, the promoter is preferably selected from among rhenium, ruthenium, platinum, palladium, boron, silver, and combinations thereof.

When the catalyst includes rhenium, the rhenium is preferably present in the catalyst in an amount between about 0.001 and about 5% by weight, more preferably between about 0.01 and about 2% by weight, most preferably between about 0.2 and about 1% by weight.

When the catalyst includes ruthenium, the ruthenium is preferably present in the catalyst in an amount between about 0.0001 and about 5% by weight, more preferably between about 0.001 and about 1% by weight, most preferably between about 0.01 and about 1% by weight.

When the catalyst includes platinum, the platinum is preferably present in the catalyst in an amount between about 0.00001 and about 5% by weight, more preferably between about 0.0001 and about 1% by weight, and most preferably between about 0.0005 and 1% by weight.

When the catalyst includes palladium, the palladium is preferably present in the catalyst in an amount between about 0.00001 and about 5% by weight, more preferably between about 0.0001 and about 2% by weight, most preferably between about 0.0.0005 and about 1% by weight.

When the catalyst includes silver, the catalyst preferably has a nominal composition including from about 0.05 to about 10 wt % silver, more preferably from about 0.07 to about 7 wt % silver, still more preferably from about 0.1 to about 5 wt % silver.

When the catalyst includes boron, the catalyst preferably has a nominal composition including from about 0.025 to about 2 wt % boron, more preferably from about 0.05 to about 1.8 wt. % boron, still more preferably from about 0.075 to about 1.5 wt % boron.

As used herein, a nominal composition is preferably a composition specified with respect to an active catalyst. The active catalyst may be either fresh or regenerated. The nominal composition may be determined by experimental elemental analysis of an active catalyst. Alternatively, the nominal composition may be determined by numerical analysis from the known amounts of catalytic metal, promoter, and support used to make the catalyst. It will be understood that the nominal composition as determined by these two methods will typically agree within conventional accuracy.

Further, as used herein, it will be understood that each of the ranges, such as of ratio or weight %, herein is inclusive of its lower and upper values.

Preparation

The present catalysts may be prepared by any of the methods known to those skilled in the art. By way of illustration and not limitation, methods of preparing a supported catalyst include impregnating a catalyst material onto the support, extruding the support material together with catalyst material to prepare catalyst extrudates, spray-drying the catalyst material and the support from a solution containing both, and/or precipitating the catalyst material onto a support. Accordingly, the supported catalysts of the present invention may be used in the form of powders, particles, pellets, monoliths, honeycombs, packed beds, foams, and aerogels. The catalyst material may include any one or combination of a catalytic metal, a precursor compound of a catalytic metal, a promoter, and a precursor compound of a promoter.

The most preferred method of preparation may vary among those skilled in the art depending, for example, on the desired catalyst particle size. Those skilled in the art are able to select the most suitable method for a given set of requirements.

One method of preparing a catalyst by impregnating a catalyst material onto a support includes impregnating the support with a solution containing the catalyst material. Suitable solvents include water and organic solvents (e.g., toluene, methanol, ethanol, and the like). Those skilled in the art will be able to select the most suitable solvent for a given catalyst material. The catalyst material may be in the form of a salt of a catalytic metal or promoter element. Thus, one method of preparing supported metal catalyst is by incipient wetness impregnation of the support with a solution of a soluble metal salt. Incipient wetness impregnation preferably proceeds by solution of a cobalt compound in a minimal amount of solvent sufficient to fill the pores of the support. Alternatively, the catalyst material may be in the form of a zero valent compound of a catalytic metal or promoter element. Thus, another preferred method is to impregnate the support with a solution of zero valent metal such as cobalt carbonyl (e.g. Co₂(CO)₈, Co₄(CO)₁₂) or the like. Multiple steps of impregnation might be necessary in order to achieve the desired amount of metal loading.

Another method of preparing a catalyst by impregnating a catalyst material onto a support includes impregnating the support with a molten salt of a catalytic metal or promoter. Thus, another method includes preparing the supported metal catalyst from a molten metal salt. One preferred method is to impregnate the support with a molten metal nitrate (e.g., Co(NO₃)₂.6H₂O). A promoter compound may be impregnated separately from any cobalt, in a separate step. Alternatively, a promoter compound may be impregnated simultaneously with, e.g. in the same solution as, at least a portion of the catalytic metal.

When a catalyst material is impregnated as a precursor of the material, e.g. a salt or zero valent compound, those skilled in the art will be able to selected the most suitable precursor.

By way of example and not limitation, suitable cobalt-containing precursor compounds include, for example, hydrated cobalt nitrate (e.g. cobalt nitrate hexadydrate), cobalt carbonyl, cobalt acetate, cobalt acetylacetonate, cobalt oxalate, and the like. Hydrated cobalt nitrate, cobalt carbonyl and cobalt acetate are exemplary of cobalt-containing precursor compounds soluble in water. Cobalt oxalate is soluble in acids or acidic solutions. Cobalt acetate and cobalt acetylacetonate are exemplary of cobalt-containing precursor compounds soluble in an organic solvent.

Suitable rhenium-containing precursor compounds soluble in water are preferred and include, for example, perrhenic acid, ammonium perrhenate, rhenium pentacarbonyl chloride, rhenium carbonyl, and the like.

Suitable ruthenium-containing precursor compounds soluble in water include for example ruthenium carbonyl, Ru(NH₃)₆.Cl₃, Ru(III)2,4-pentanedionoate, ruthenium nitrosyl nitrate, and the like. Water-soluble ruthenium-containing precursor compounds are preferred.

Suitable platinum-containing precursor compounds soluble in water include, for example, Pt(NH₃)₄(NO₃)₂ and the like. Alternatively, the platinum-containing precursor may be soluble in an organic solvent, such as platinum acetyl acetonate soluble in acetone.

Suitable boron-containing precursor compounds soluble in water include, for example, boric acid, and the like. Alternatively, the boron-containing precursor may be soluble in an organic solvent.

Suitable silver-containing precursor compounds soluble in water include, for example, silver nitrate (AgNO₃) and the like. Alternatively, the silver-containing precursor may be soluble in an organic solvent.

Suitable palladium-containing precursor compounds include palladium nitrate (Pd(NO₃)₂) and the like. Suitable palladium-containing precursor compounds soluble in an organic solvent include palladium dioxide (PdO₂), which is soluble in acetone, and the like.

The impregnated support is preferably treated to form a treated impregnated support. The treatment may include drying the impregnated support. Drying the impregnated support preferably occurs at a temperature between 80 and 150° C. Typically, drying proceeds for from 0.5 to 24 hours at a pressure of from about 1 to about 75 atm, more preferably from about 1 to about 10 atm, most preferably at about 1 atm.

Alternatively, or in combination, treating an impregnated support to form a treated impregnated support may include calcining the impregnated support. The calcination preferably achieves oxidation of any impregnated compound or salt of a supported material to an oxide compound of the supported material. When the catalytic metal includes cobalt, the calcination preferably proceeds at a temperature of at least 200° C. Further, the calcination preferably proceeds at a temperature less than the temperature at which loss of support surface area is appreciable. It is believed that at temperatures above 900° C. loss of support surface area is appreciable. Typically, calcining proceeds from 0.5 to 24 hours at a pressure of 1 to 75 atm, more preferably 1-10 atm, most preferably at 1 atm.

The impregnation of catalytic metal and any optional promoter on a support may proceed by multistep impregnation, such as by two, three, or four impregnation steps. Each impregnation step may include impregnation of any one or combination of catalytic metal and promoter. Each impregnation step may be followed by any of the above-described treatments of the impregnated support. In particular, each step of impregnating the support to form an impregnated support may be followed by treating the impregnated support to form a treated impregnated support. Thus, a multistep impregnation may include multiple steps of drying and/or calcination. Each subsequent step of drying may proceed at a different temperature from any earlier steps of drying. Further, each subsequent step of calcination may proceed at a different temperature from any earlier steps of calcination. By way of example and not limitation, a multi-step impregnation may include calcining the support at a first temperature that is higher than the temperature for subsequent calcinations.

Typically, at least a portion of the metal(s) of the catalytic metal component of the catalysts of the present invention is present in a reduced state (i.e., in the metallic state). Therefore, it is normally advantageous to activate the catalyst prior to use by a reduction treatment in the presence of a reducing gas at an elevated temperature. The reducing gas preferably includes hydrogen. Typically, the catalyst is treated with hydrogen or a hydrogen-rich gas at a temperature in the range of from about 75° C. to about 500° C., for about 0.5 to about 50 hours at a pressure of about 1 to about 75 atm. Pure hydrogen may be used in the reduction treatment, as may a mixture of hydrogen and an inert gas such as nitrogen, or a mixture of hydrogen and other gases as are known in the art, such as carbon monoxide and carbon dioxide. Reduction with pure hydrogen and reduction with a mixture of hydrogen and carbon monoxide are preferred. The amount of hydrogen may range from about 1% to about 100% by volume. rich gas at a temperature in the range of from about 75° C. to about 500° C., for about 0.5 to about 50 hours at a pressure of about 1 to about 75 atm. Pure hydrogen may be used in the reduction treatment, as may a mixture of hydrogen and an inert gas such as nitrogen, or a mixture of hydrogen and other gases as are known in the art, such as carbon monoxide and carbon dioxide. Reduction with pure hydrogen and reduction with a mixture of hydrogen and carbon monoxide are preferred. The amount of hydrogen may range from about 1% to about 100% by volume.

Feed Gas

The feed gases charged to the process of the invention comprise hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, partial oxidation, or other processes known in the art. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than 0.5:1 (e.g., from about 0.67 to 2.5). Preferably, when cobalt, nickel, and/or ruthenium catalysts are used, the feed gas stream contains hydrogen and carbon monoxide in a molar ratio of from about 1.7:1 to about 2.2:1. Preferably, when iron catalysts are used the feed gas stream contains hydrogen and carbon monoxide in a molar ratio between about 1.6:1 and 2.1:1. The feed gas may also contain carbon dioxide. The feed gas stream should contain a low concentration of compounds or elements that have a deleterious effect on the catalyst, such as poisons. For example, the feed gas may need to be pretreated to ensure that it contains low which they are to be used. The activation of the catalyst may be performed in the same or a different reactor.

The Fischer-Tropsch process is typically run in a continuous mode. In this mode, the gas hourly space velocity through the reaction zone typically may range from about 50 to about 10,000 hr⁻¹, preferably from about 300 hr⁻¹ to about 2,000 hr⁻¹. The gas hourly space velocity is defined as the volume of reactants per time per reaction zone volume. The volume of reactant gases is at standard conditions (standard pressure of 1 atm (101 kPa) and standard temperature of 0° C. (273.16 K)). The reaction zone volume is defined by the portion of the reaction vessel volume where reaction takes place and which is occupied by a gaseous phase comprising reactants, products and/or inerts; a liquid phase comprising liquid/wax products and/or other liquids; and a solid phase comprising catalyst. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psia (552 kPa) to about 1000 psia (6895 kPa), more preferably from 80 psia (552 kPa) to about 600 psia (4137 kPa), and still more preferably, from about 140 psia (965 kPa) to about 500 psia (3447 kPa).

The products resulting from the process will have a great range of molecular weights. Typically, the carbon number range of the product hydrocarbons will start at methane and continue to about 50 to 100 carbons or more per molecule as measured by current analytical techniques. The process is particularly useful for making hydrocarbons having five or more carbon atoms especially when the above-referenced preferred space velocity, temperature and pressure ranges are employed.

The wide range of hydrocarbons produced in the reaction zone will typically afford liquid phase products at the reaction zone operating conditions. Therefore the effluent stream of the reaction zone will often be a mixed phase stream including liquid and gas phase products. The effluent gaseous stream of the reaction zone may be cooled to condense additional amounts of hydrocarbons and passed into a vapor-liquid separation zone separating the liquid and vapor phase products. The gaseous material may be passed into a second stage of cooling for recovery of additional hydrocarbons. The liquid material from the reaction zone together with any liquid from a subsequent separation zone may be fed into a fractionation column. Typically, a stripping column is employed first to remove light hydrocarbons such as propane and butane. The remaining hydrocarbons may be passed into a fractionation column where they are separated by boiling point range into products such as naphtha, kerosene and fuel oils. Hydrocarbons recovered from the reaction zone and having a boiling point above that of the desired products may be passed into conventional processing equipment such as a hydrocracking zone in order to reduce their molecular weight down to desired products such as middle distillates and gasoline. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. The following specific embodiments are to be construed as illustrative and not as constraining the scope of the present invention in any way whatsoever.

### EXAMPLES

General Analytical Procedure

The uncondensed gaseous products from the reactors were analyzed using a common on-line HP Refinery Gas Analyzer. The Refinery Gas Analyzer was equipped with two thermal conductivity detectors and measured the concentrations of CO, H₂, N₂, CO₂, CH₄, C₂ to C₅ alkenes/alkanes/isomers and water in the uncondensed reactor products.

The products from each of the hot and cold traps were separated into an aqueous and an organic phase. The organic phase from the hot trap was usually solid at room temperature. A portion of this solid product was dissolved in carbon disulfide before analysis. The organic phase from the cold trap was usually liquid at room temperature and was analyzed as obtained. The aqueous phase from the two traps was combined and analyzed for alcohols and other oxygenates.

Two off-line gas chromatographs equipped with flame ionization detectors were used for the analysis of the organic and aqueous phases collected from the wax and cold traps.

General Procedure for Batch Testing

For the batch tests, a 2 mL pressure vessel was heated at 225° C. under 1000 psig (6994 kPa) of H₂:CO (2:1) and maintained at that temperature and pressure for 1 hour. In a typical run, roughly 20 mg of the reduced catalyst and 1 mL of n-octane was added to the vessel. After one hour, the reactor vessel was cooled in ice, vented, and an internal standard of di-n-butylether was added. The reaction product was analyzed on an HP6890 gas chromatograph. Hydrocarbons in the range of C₁₁-C₄₀ were analyzed relative to the internal standard. The lower hydrocarbons were not analyzed, since they are masked by the solvent and are also vented as the pressure is reduced. In the following examples, all catalyst having the same number designations but different letter designations were made on the same day and tested in the synthesis reaction under the same conditions.

A nominal composition was computed according to the amount by weight of alumina or fluorided alumina, the amount of elemental cobalt, the amount of any elemental rhenium, and the amount of any elemental promoter used to prepare the catalyst. Where a % is used in the nominal composition, it is a weight %.

A C₁₁₊ Productivity (g C₁₁₊/hour/kg catalyst) was calculated based on the integrated production of the C₁₁-C₄₀ hydrocarbons per kg of catalyst per hour. The logarithm of the weight fraction for each carbon number ln(Wₙ/n) was plotted as the ordinate vs. number of carbon atoms in (Wₙ/n) as the abscissa. From the slope, a value of a was obtained. The results of runs over a variety of catalysts at 225° C. are shown in Tables 1-3. O/P indicates olefin/paraffin ratio.

### Comparative Example A

ZrO₂ (1 g) was slurried into a solution of tetracobaltdodecacarbonyl (0.5 g) in a minimum volume of dry toluene. The slurry was stirred well for 10 mins, then evaporated to dryness at a low temp. The recovered dry sample was heated in flowing hydrogen, using a water trap. For the heating, the temperature was raised to 200° C. at a rate of 10° C./min and held there for 30 mins. The sample was then cooled and flushed with nitrogen to obtain a catalyst with a nominal composition of 20%Co/ZrO₂. The catalyst was tested using the general procedure for batch tests described above. Results of testing are given in Table 1, and reproduced in Tables 2 and 3

### Examples 1-7

### Silica-Zirconia Support

These examples demonstrate an improvement in productivity ranging from 10% to 60% for various cobalt-based Fischer-Tropsch catalysts each using a silica-zirconia support as compared to a corresponding catalyst using a zirconia support.

Further, these examples demonstrate an improvement in productivity ranging from 0% to 70% for various cobalt-based Fischer-Tropsch catalysts each using a silica-zirconia support as compared to a corresponding catalyst using a zirconia support.

TABLE 1

| | Catalyst Nominal | | | | | | |
|---|---|---|---|---|---|---|---|
| Exam- | Composition (wt. %) | | | | C₁₁⁺ | | O/P |
| ple | Co | Ru | Pt | Support | productivity | α | (C₁₁⁺) |
| A | 20 | | | ZrO₂ | 100 | 0.85/0.9 | 0.76 |
| 1 | 20 | | | SiO₂—ZrO₂ | 70 | 0.9 | 0.94 |
| 2 | 20 | | | SiO₂—ZrO₂ | 160 | 0.9 | 0.58 |
| 3 | 20 | | | SiO₂—ZrO₂ | 160 | 0.89 | 0.38 |
| 4 | 20 | | | SiO₂—ZrO₂ | 150 | 0.87 | 0.12 |
| 5 | 20 | 0.25 | | SiO₂—ZrO₂ | 160 | 0.9 | 0.75 |
| 6 | 20 | 0.5 | | SiO₂—ZrO₂ | 110 | 0.89 | 0.40 |
| 7 | 25 | | 0.1 | SiO₂—ZrO₂ | 120 | 0.88 | 0.53 |

### Example 1

SiO₂—ZrO₂ (7.9500 g, Aldrich) was slurried into molten Co(NO₃)₂.6H₂O (9.8768 g). The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/SiO₂—ZrO₂. The catalyst was tested using the general for batch tests described above. Results of testing are given in Table 1.

### Example 2

SiO₂—ZrO₂ (8.0000 g, Aldrich) was slurried into molten Co(NO₃)₂.6H₂O (9.8768 g). The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/SiO₂—ZrO₂. The catalyst was tested using the general procedure for batch tests described above. Results of testing are given in Table 1.

### Example 3

SiO₂—ZrO2 (1 g, Aldrich) was slurried into a solution of tetracobaltdodecacarbonyl (0.5 g) in a minimum volume of dry toluene. The slurry was stirred well for 10 mins, then evaporated to dryness at a low temp. The recovered dry sample was heated in flowing hydrogen, using a water trap. For the heating, the temperature was raised to 200° C. at a rate of 10° C./min and held there for 30 mins. The sample was then cooled and flushed with nitrogen to obtain a catalyst with a nominal composition of 20%Co/SiO₂—ZrO₂. The catalyst was tested using the general procedure for batch tests described above. Results of testing are given in Table 1.

### Example 4

SiO₂—ZrO2 (1 g, Aldrich) was slurried into a solution of tetracobaltdodecacarbonyl (0.5 g) in a minimum volume of dry toluene. The slurry was stirred well for 10 mins, then evaporated to dryness at a low temp. The recovered dry sample was calcined at 600° C., cooled, then heated in flowing hydrogen, using a water trap. For the heating, the temperature was raised to 200° C. at a rate of 10° C./min and held there for 30 mins. The sample was then cooled and flushed with nitrogen to obtain a catalyst with a nominal composition of 20%Co/SiO₂—ZrO₂. The catalyst was tested using the general procedure for batch tests described above. Results of testing are given in Table 1.

### Example 5

Co(NO₃)₂.6H₂O (9.8768 g) was melted and thoroughly mixed with a solution of Ru(III)2,4-pentantdionate (0.0985 g) in a small amount of CH₃CN. SiO₂—ZrO₂ (7.9750 g, Aldrich) was slurried into the mixed solution. The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/0.25%Ru/SiO₂—ZrO₂. The catalyst was tested using the general procedure for batch tests described above. Results of testing are given in Table 1.

### Example 6

Co(NO₃)₂.6H₂O (9.8768 g) was melted and thoroughly mixed with a solution of Ru(III)2,4-pentantdionate (0.1971 g) in a small amount of CH₃CN. SiO₂—ZrO₂ (8.00 g Aldrich) was slurried into the mixed solution. The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/0.5%Ru/SiO₂—ZrO₂. The catalyst was tested using the general procedure for batch tests described above. Results of testing are given in Table 1.

### Example 7

SiO₂—ZrO₂ (30 g, Engelhard #712A-6-234-1) was treated in a rotary evaporator at 70° C. with an aqueous solution containing Co(NO₃).6H₂O (24 g) followed by calcination at 250° C. in 1.5 L/min air. This material was then treated in a rotary evaporator at 70° C. with an aqueous solution containing Co(NO₃).6H₂O (21 g) and Pt(NH₃)₄(NO₃)₂ (30 mg) followed by calcination at 250° C. in 1.5 L/min air to obtain a catalyst with a nominal composition of 25%Co/0.1%Pt/SiO₂—ZrO₂. The catalyst was tested using the general procedure for batch tests described above. of testing are given in Table 1.

### Examples 8-12

### Sulfated Zirconia

These examples demonstrate an improvement in productivity ranging from 10%-30% for various cobalt-based Fischer-Tropsch catalysts each using a sulfated zirconia support as compared to a corresponding catalyst using a zirconia support.

In the examples below, SO₄—ZrO₂ (Engelhard) indicates a sulfated zirconia hydrate nominally 3-10% SO₄²⁻ which was obtained from Engelhard, treated by calcination at 500° C. for 5 hours in 1.5 L/min air, cooled, and stored for later use.

TABLE 2

| | Catalyst Nominal | | | | | C₁₁⁺ | | |
|---|---|---|---|---|---|---|---|---|
| Exam- | Composition (wt. %) | | | | | produc- | | O/P |
| ple | Co | Ru | Re | Ag | Support | tivity | α | (C₁₁ ⁺) |
| A | 20 | | | | ZrO₂ | 100 | 0.85/0.9 | 0.76 |
| 8 | 16 | | | | SO₄—ZrO₂ | 130 | 0.88 | 0.70 |
| 9 | 20 | | | | SO₄—ZrO₂ | <1 | | |
| 10 | 16 | 0.1 | | | SO₄—ZrO₂ | 120 | 0.87 | 0.67 |
| 11 | 16 | | 1 | | SO₄—ZrO₂ | 130 | 0.88 | 0.97 |
| 12 | 15 | | | 2.5 | SO₄—ZrO₂ | 110 | 0.86 | 0.49 |

### Example 8

SO₄—ZrO₂ (8.0000 g, Engelhard) was slurried into molten Co(NO₃)₂.6H₂O (9.9 g). The sample was evaporated and dried at 110° C. The recovered dry sample was calcined at 500° C. in air for 5 hours. The sample was then reduced at 500° C. for 16 hours and cooled to obtain a catalyst with a nominal composition of 16%Co/SO₄—ZrO₂. The catalyst was tested using the general procedure for batch tests described above. Results of testing are given in Table 2.

### Example 9

SO₄—ZrO₂ (8.0000 g, Engelhard) was slurried into molten Co(NO₃)₂.6H₂O (9.8768 g). The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/SO₄—ZrO₂. The catalyst was tested using the general procedure for batch tests described above. Results of testing are given in Table 2.

### Example 10

Co(NO₃)₂.6H₂O (10 g) was melted and thoroughly mixed with a solution of a ruthenium nitrosyl nitrate (0.2 g) in a small amount of CH₃CN. SO₄—ZrO₂ (8.0 g, Engelhard) was slurried into the mixed solution. The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/0.1%Ru/SO₄—ZrO₂. The catalyst was tested using the general procedure for batch tests described above. Results of testing are given in Table 2.

### Example 11

Co(NO₃)₂.6H₂O (9.9 g) was melted and thoroughly mixed with a solution of perrhenic acid (1 g) in a small amount of water. SO₄—ZrO₂ (8.0 g, Engelhard) was slurried into the mixed solution. The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/1%Re/SO₄—ZrO₂. The catalyst was tested using the general procedure for batch tests described above. Results of testing are given in Table 2.

### Example 12

SO₄—ZrO₂ (28.8 g, Engelhard) was treated in a rotary evaporator at 70° C. with an aqueous solution containing Co(NO₃).6H₂O (23 g) followed by calcination at 250° C. in 1.5 L/min air. A portion of this material (8 g) was treated in a rotary evaporator at 70° C. with an aqueous solution containing AgNO₃ (0.32 g) followed by calcination at 250° C. in 1.5 L/min air to obtain a catalyst with a nominal composition of 15%Co/2.5%Ag/SO₄—ZrO₂. The catalyst was tested using the general procedure for batch tests described above. Results of testing are given in Table 2.

### Examples 13-27

### Tungstated Zirconia

These examples demonstrate an improvement in productivity of up to 30% for cobalt-based based Fischer-Tropsch catalysts using a tungstated support, as compared to a corresponding catalyst using a zirconia support.

TABLE 3

| | | | | | | | | C₁₁⁺ | | |
|---|---|---|---|---|---|---|---|---|---|---|
| Exam- | Catalyst Nominal Composition (wt. %) | | | | | | | produc- | | O/P |
| ple | Co | Ru | Re | Pt | Ag | La | Support | tivity | α | (C₁₁⁺) |
| A | 20 | | | | | | ZrO₂ | 100 | 0.85/ | 0.76 |
| | | | | | | | | | 0.9 | |
| 13 | 20 | | | | | | WO₃-ZrO₂ | <1 | | |
| 14 | 20 | | | | | | WO₃-ZrO₂ | 130 | 0.91 | 0.58 |
| 15 | 20 | 0.25 | | | | | WO₃-ZrO₂ | 10 | .85 | |
| 16 | 20 | 0.5 | | | | | WO₃-ZrO₂ | 20 | 0.9 | |
| 17 | 25 | | | | | 23.5 | WO₃-ZrO₂ | 50 | 0.87 | 0.99 |
| 18 | 25 | | | 0.05 | | | WO₃-ZrO₂ | 80 | 0.89 | 0.80 |
| 19 | 15 | | | | 2 | | WO₃-ZrO₂ | 40 | 0.87 | 0.69 |
| 20 | 25 | | | | 2 | | WO₃-ZrO₂ | 60 | 0.88 | 0.27 |
| 21 | 25 | 0.1 | | 0.05 | | | WO₃-ZrO₂ | 60 | 0.87 | 0.83 |
| 22 | 25 | 0.1 | | 0.05 | | | WO₃-ZrO₂ | 60 | 0.88 | 0.99 |
| 23 | 15 | 0.1 | | | 2 | | WO₃-ZrO₂ | 50 | 0.87 | 0.86 |
| 24 | 15 | 0.1 | | | 2 | | WO₃-ZrO₂ | 70 | 0.8 | 0.80 |
| 25 | 25 | | | 0.05 | 2 | | WO₃-ZrO₂ | 70 | 0.89 | 0.91 |
| 26 | 25 | | 1 | 0.05 | | | WO₃-ZrO₂ | 80 | 0.89 | 0.73 |
| 27 | 25 | | 1 | 0.05 | 2 | | WO₃-ZrO₂ | 40 | 0.87 | 0.03 |

### Example 13

WO₃—ZrO₂ (7.9500 g, Engelhard) was slurried into molten Co(NO₃)₂.6H₂O (9.8768 g). The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/WO₃—ZrO₂. The catalyst was tested using the general procedure for batch tests described above. Results of testing are given in Table 3.

### Example 14

WO₃—ZrO2 (1 g, Engelhard) was slurried into a solution of tetracobaltdodecacarbonyl (0.5 g) in a minimum volume of dry toluene. The slurry was stirred well for 10 mins, then evaporated to dryness at a low temp. The recovered dry sample was heated in flowing hydrogen, using a water trap. For the heating, the temperature was raised to 200° C. at a rate of 10° C./min and held there for 30 mins. The sample was then cooled and flushed with nitrogen to obtain a catalyst with a nominal composition of 20%Co/WO₃—ZrO₂. The catalyst was tested using the general procedure for batch tests described above. Results of testing are given in Table 3.

### Example 15

Co(NO₃)₂.6H₂O (9.8768 g) was melted and thoroughly mixed with a solution of Ru(III)2,4-pentantdionate (0.0985 g) in a small amount of CH₃CN. WO₃—ZrO₂ (7.9750 g, Engelhard) was slurried into the mixed solution. The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/0.25%Ru/WO₃—ZrO₂. The catalyst was tested using the general procedure for batch tests described above. Results of testing are given in Table 3.

### Example 16

Co(NO₃)₂.6H₂O (9.8768 g) was melted and thoroughly mixed with a solution of Ru(III)2,4-pentantdionate (0.1971 g) in a small amount of CH₃CN. WO₃—ZrO₂ (8.00 g, Engelhard) was slurried into the mixed solution. The sample was evaporated and dried at 110° C. The recovered dry sample was calcined at 500° C. in air for 5 hours. The sample was then reduced at 500° C. for 16 hours and cooled to obtain a catalyst with a nominal composition of 20%Co/0.5%Ru/WO₃—ZrO₂. The catalyst was tested using the general procedure for batch tests described above. Results of testing are given in Table 3.

### Example 17

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (4 g) was then treated in a rotary evaporator at 70° C. with an aqueous solution containing Co(NO₃).6H₂O (2.8 g) and La(NO₃)₃).6H₂O (2.9 g) followed by calcination at 250° C. in 1.5 L/min air to obtain a catalyst with a nominal composition of 25%Co/23.5%La/WO₃—ZrO₂. The catalyst was tested using the general procedure for batch tests described above. Results of testing are given in Table 3.

### Example 18

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (25 g) was treated in a rotary evaporator at 70° C. with an aqueous solution containing Co(NO₃).6H₂O (17.5 g) and Pt(NH₃)₄(NO₃)₂ (25 mg) followed by calcination at 250° C. in 1.5 L/min air to obtain a catalyst with a nominal composition of 25%Co/0.05%Pt/WO₃—ZrO₂. The catalyst was tested using the general procedure for batch tests described above. Results of testing are given in Table 3.

### Example 19

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (20 g) was treated in a rotary evaporator at 70° C. with an aqueous solution containing AgNO₃ (0.62 g) followed by calcination at 250° C. in 1.5 L/min air to obtain a catalyst with a nominal composition of 15%Co/2%Ag/WO₃—ZrO₂. The catalyst was tested using the general procedure for batch tests described above. Results of testing are given in Table 3.

### Example 20

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (20 g) was treated in a rotary evaporator at 70° C. with an aqueous solution containing AgNO₃ (0.62 g) followed by calcination at 250° C. in 1.5 L/min air. A portion of this material (2 g) was treated in a rotary evaporator at 70° C. with an aqueous solution containing Co(NO₃).6H₂O (1.4 g) followed by calcination at 250° C. in 1.5 L/min air to obtain a catalyst with a nominal composition of 25%Co/2%Ag/WO₃—ZrO₂. The catalyst was tested using the general procedure for batch tests described above. Results of testing are given in Table 3.

### Example 21

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (25 g) was treated in a rotary evaporator at 70° C. with an aqueous solution containing Co(NO₃).6H₂O (17.5 g) and Pt(NH₃)₄(NO₃)₂ (25 mg) followed by calcination at 250° C. in 1.5 L/min air. A portion of this material (4 g) was treated in a rotary evaporator at 70° C. with an aqueous solution containing RuCl₃ (8 mg) followed by calcination at 250° C. in 1.5 L/min air to obtain a catalyst with a nominal composition of 25%Co/0.1%Ru/0.05%Pt/WO₃—ZrO₂. The catalyst was tested using the general procedure for batch tests described above. Results of testing are given in Table 3.

### Example 22

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (25 g) was treated in a rotary evaporator at 70° C. with an aqueous solution containing Co(NO₃).6H₂O(17.5 g) and Pt(NH₃)₄(NO₃)₂ (25 mg) follow calcination at 250° C. in 1.5 L/min air. A portion of this material (1 g) was treated in a rotary evaporator at 70° C. with an acetone solution containing RuCl₃ (4 mg) followed by calcination at 250° C. in 1.5 L/min air to obtain a catalyst with a nominal composition of 25%Co/0.1%Ru/0.05%Pt/WO₃—ZrO₂. The catalyst was tested using the general procedure for batch tests described above. Results of testing are given in Table 3.

### Example 23

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (20 g) was treated in a rotary evaporator at 70° C. with an aqueous solution containing AgNO₃ (0.62 g) followed by calcination at 250° C. in 1.5 L/min air. A portion of this material (4 g) was treated in a rotary evaporator at 70° C. with an acetone solution containing RuCl₃ (8 mg) to obtain a catalyst with a nominal composition of 15%Co/0.1%Ru/2%Ag/WO₃—ZrO₂. The catalyst was tested using the general procedure for batch tests described above. Results of testing are given in Table 3.

### Example 24

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (20 g) was treated in a rotary evaporator at 70° C. with an aqueous solution containing AgNO₃ (0.62 g) followed by calcination at 250° C. in 1.5 L/min air. A portion of this material (1 g) was treated in a rotary evaporator at 70° C. with an acetone solution containing RuCl₃ (4 mg) to obtain a catalyst with a nominal composition of 15%Co/0.1%Ru/2%Ag/WO₃—ZrO₂. The catalyst was tested using the general procedure for batch tests described above. Results of testing are given in Table 3.

### Example 25

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (25 g) was treated in a rotary evaporator at 70° C. with an aqueous solution containing Co(NO₃).6H₂O (17.5 g) and Pt(NH₃)₄(NO₃)₂ (25 mg) followed by calcination at 250° C. in 1.5 L/min air. A portion of this material (4 g) was treated in a rotary evaporator at 70° C. with an aqueous solution containing AgNO₃ (0.124 g) followed by calcination at 250° C. in 1.5 L/min air to obtain a catalyst with a nominal composition of 25%Co/0.05%Pt/2%Ag/WO₃—ZrO₂. The catalyst was tested using the general procedure for batch tests described above. Results of testing are given in Table 3.

### Example 26

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (25 g) was treated in a rotary evaporator at 70° C. with an aqueous solution containing Co(NO₃).6H₂O (17.5 g) and Pt(NH₃)₄(NO₃)₂ (25 mg) followed by calcination at 250° C. in 1.5 L/min air. A portion of this material (4 g) was treated in a rotary evaporator at 70° C. with an aqueous solution containing Re₂O₇ (52 mg) followed by calcination at 250° C. in 1.5 L/min air to obtain a catalyst with a nominal composition of 25%Co/1%Re/0.05%Pt/WO₃—ZrO₂. The catalyst was tested using the general procedure for batch tests described above. Results of testing are given in Table 3.

### Example 27

WO₃—ZrO₂ (50 g, Engelhard #712A-6-242-1) was treated in a rotary evaporator at 70° C. with an aqueous solution containing Co(NO₃).6H₂O (40 g) followed by calcination at 250° C. in 1.5 L/min air. A portion of this material (25 g) was treated in a rotary evaporator at 70° C. with an aqueous solution containing Co(NO₃).6H₂O (17.5 g) and Pt(NH₃)₄(NO₃)₂ (25 mg) followed by calcination at 250° C. in 1.5 L/min air. A portion of this material (4 g) was treated in a rotary evaporator at 70° C. with an aqueous solution containing Re₂O₇ (52 mg) followed by calcination at 250° C. in 1.5 L/min air. A portion of this material (1 g) was treated in a rotary evaporator at 70° C. with an aqueous solution containing AgNO₃ (31 mg) followed by calcination at 250° C. in 1.5 L/min to obtain a catalyst with a nominal composition of 25%Co/1%Re/0.05%Pt/2%Ag/WO₃—ZrO₂. The catalyst was tested using the general procedure for batch tests described above. Results of testing are given in Table 3.

While preferred embodiments of this invention have been shown and described, modifications thereof can be made by one skilled in the art without departing from the spirit or teaching of this invention. The embodiments described herein are exemplary only and are not limiting. Many variations and modifications of the catalyst and process are possible and are within the scope of the invention. Accordingly, the scope of protection is not limited to the embodiments described herein.

What is claimed is:

1. A process for producing hydrocarbons, comprising:
converting a feed stream comprising carbon monoxide and hydrogen to a product stream comprising hydrocarbons in the presence of a catalyst,
wherein the catalyst comprises:
cobalt; and
a modified zirconia support selected from the group consisting of silica-zirconia, sulfated zirconia, and tungstated zirconia.

2. The process according to claim 1 wherein the catalyst has an improved performance with respect to a corresponding catalyst comprising a zirconia support.

3. The process according to claim 1 wherein the improvement in performance is at least about 5%.

4. The process according to claim 3 wherein the improvement in performance is at least about 10%.

5. The process according to claim 2 wherein the performance comprises the productivity.

6. The process according to claim 5 wherein the product stream comprises C₁₁₊ hydrocarbons.

7. The process according to claim 6 wherein the improvement in performance is at least about 5%.

8. The process according to claim 7 wherein the improvement in performance is at least about 10%.

9. The process according to claim 2 wherein the performance comprises the ratio of paraffins to olefins in the hydrocarbon stream.

10. The process according to claim 9 wherein the improvement in performance is at least about 5%.

11. The process according to claim 9 wherein the product stream comprises C₁₁⁺ hydrocarbons.

12. The process according to claim 1 wherein the catalyst has an improved property with respect to a corresponding catalyst comprising a zirconia support.

13. The process according to claim 12 wherein the property comprises acidity.

14. The process according to claim 12 wherein the property comprises dispersion.

15. The process according to claim 12 wherein the property comprises reducibility.

16. The process according to claim 1 wherein the catalyst further comprises a promoter selected from the group consisting of rhenium, ruthenium, platinum, silver, lanthanum, and combinations thereof.

17. The process according to claim 1 wherein the catalysts comprises between about 2 and about 35% cobalt by weight of the total catalyst.

18. A process for producing hydrocarbons, comprising:
converting a feed stream comprising carbon monoxide and hydrogen to a product stream comprising hydrocarbons in the presence of a catalyst,
wherein the catalyst comprises:
a modified zirconia support selected from the group consisting of silica-zirconia, sulfated zirconia, and tungstated zirconia; and
cobalt in an amount between about 10 percent by weight of the total catalyst and about 50 percent by weight of the total catalyst;
wherein the catalyst has an improved performance with respect to a corresponding catalyst comprising a zirconia support.

19. The process according to claim 18 wherein the improvement is performance is at least about 10%.

20. The process according to claim 19 wherein the improvement in performance is at least about 30%.

21. A process for producing hydrocarbons comprising contacting a feed stream comprising hydrogen and carbon monoxide with a catalyst in a reaction zone maintained at conversion-promoting conditions effective to produce an effluent stream comprising hydrocarbons wherein the catalyst comprises:
a modified zirconia support selected from the group consisting of silica-zirconia, sulfated zirconia, and tungstated zirconia;
a first metal comprising cobalt in an amount between about 10 percent by weight of the total catalyst and about 50 percent by weight of the total catalyst; and
a second metal selected from the group consisting of rhenium, ruthenium, platinum, silver, and lanthanum.
